(19) United States
(12) Patent Application Publication
(10) Pub. No.: US 2003/0175318 A1
(43) Pub. Date: Sep. 18, 2003

(54) Application of germination solution improved efficacy of biological decontamination

(76) Inventors: Amanda S. Schilling, Fredericksburg, VA (US); Angela G. Mersiowsky, King George, VA (US); Susan M. Thomas, King George, VA (US); Jerry S. Brown, Woodford, VA (US); Brian Williamson, Manassas, VA (US); Angel A. Fitzgerald, Fredericksburg, VA (US); Shelia M. Kuhstoss, Fredericksburg, VA (US); Michael Walter, King George, VA (US)

Correspondence Address: James B. Bechtel, Esq., NSWCDD (XDC1), Dahlgren, VA, 22448-5100, US

(21) Appl. No.: 10/090,798

(22) Filed: Mar. 6, 2002

(51) Int. Cl. A01N 25/00; A61K 33/40; A01N 59/06

(52) U.S. Cl. 424/405; 424/613; 424/682

(57) ABSTRACT

Decontamination of biological spores occurs by applying a germination solution for spores containing calcium ions, preferably calcium chloride, and dipicolinic acid in combination with a decontaminant. The germination composition may include water and a surfactant. The germination solution allows for lower concentrations of select decontaminants to be effectively used against spore contamination. The germination composition may be applied prior to, concurrently, or sequentially with repeated applications of the decontaminant.

### STATEMENT REGARDING FEDERALLY SPONSORED RESEARCH OR DEVELOPMENT

The invention described herein may be manufactured and used by or for the government of the United States of America for governmental purposes without the payment of any royalties thereon or therefor.

### BACKGROUND OF THE INVENTION

1. Field of the Invention

The present invention provides a method and composition for decontaminating bacteria contamination, and product thereby, by applying a spore germination solution containing calcium chloride (CaCl₂) and dipicolinic acid (DPA) in combination with a decontaminant, such as a disinfectant. The germination composition preferably includes water and one or more surfactants. This germination solution allows for lower concentrations of selected disinfectants to be effectively used against spore contamination. The germination composition improves the efficacy of select decontaminants. The germination composition may be applied prior to, concurrently or sequentially with repeated applications of disinfectant.

2. Brief Description of the Related Art

Disinfecting areas containing spore contamination generally requires the extensive use of harsh detergents or other cleaners to ensure the spores are neutralized. The resilience of spores has necessitated such inventions as described in U.S. Pat. No. 5,795,730 to Tautvydas, which describes a biological indicator system that determines the effectiveness of a sterilization process by first contacting contaminate spores with a sterilant and then utilizing a germination media and calculating the germination rate and spore viability. Although used to test the effectiveness of a sterilant, Tautvydas does not utilize the germination media in a manner to increase the effectiveness of the sterilant against the spores.

### SUMMARY OF THE INVENTION

The present invention includes a method for decontaminating contamination containing biological spores comprising the steps of contacting the contamination with a spore germination composition containing calcium ion, particularly supplied by calcium chloride, and dipicolinic acid effective to cause germination of the spores and applying a decontaminating solution to kill the germinated spores. The germination composition may be applied prior to, concurrently or sequentially with the application of the decontaminating solution.

The present invention also includes a germination composition for decontaminating biological spores comprising dipicolinic acid and calcium ion, such as that supplied by calcium chloride, which may further include water and/or a surfactant.

Additionally, the present invention includes a decontaminated surface, such as a hard surface, made by the process comprising the steps of contacting a surface with a spore germination composition comprising dipicolinic acid and calcium ion effective to cause germination of the spores and applying a decontaminating solution to kill the germinated spores.

### DESCRIPTION OF THE DRAWING

FIG. 1 shows the logarithmic reduction in 10⁸ Bacillus globigii colony forming units per milliliter (CFU/ml) after 15 minute exposure to candidate decontamination solutions.

### DETAILED DESCRIPTION OF THE PREFERRED EMBODIMENT

The present invention provides a composition for decontaminating bacterial contamination, method of decontaminating spore contamination with the composition, and product thereby, by applying a germination solution for spores in combination with a decontaminant. The germination composition includes calcium ions and dipicolinic acid, with calcium chloride preferably used to supply the calcium ions. Preferably, water and one or more surfactants are added to the germination solution. This germination solution allows for lower concentrations of selected decontaminants to be effectively used against spore contamination, by improving the efficacy of the decontaminants. The germination composition may be applied prior to or concurrently with the application of the decontaminant.

The method of the present invention for decontaminating biological spore contamination uses an amount of spore germination composition, over a sufficient time period, to cause germination of the biological spores. With the spores germinated within a contaminated area select disinfectants show increased effectiveness against the spores. Upon germinating, the spore cortex breaks down, losing heat and chemical resistance, which results in increased susceptibility to being killed by the decontaminant.

Application of the spore germination composition that is effective to cause germination of the spores includes amounts of particular germination compositions under applicable conditions for a measurable kill ratio of spores due to the germination process of those spores. As such, variables include the type and strength of the germination composition, the type and amount of spores within a contaminated area, the efficiency of contact between the spores and the germination composition, such as germination composition applications, the area or article contaminated, and other like factors and conditions that affect the application of the spore germination composition against a given spore contamination, with such factors and conditions determinable by one of ordinary skill in the art of spore decontamination.

Upon germination, the biosynthetic activity of the bacteria resumes and a rod shaped cell is regenerated. Once germination is initiated the spores are committed to germination. The spore becomes committed to germination before any morphological changes are evident. Germination is characterized by the degradation of the spore as identified by the breakdown of the cortex, release of dipicolinic aid (DPA), calcium and other ions, and the intake of water. Bacillus subtilis spores germinate within from about twenty to about thirty minutes from the addition of the germinants to RNA synthesis, with this time period widely variable even within a single spore population. When spores germinate, they lose heat and chemical resistance because of the degradation of the spore cortex, disruption of the spore coat, and the rehydration of the spore core. The loss of heat resistance is one of the earliest signs of germination.

After the first minute of germination, the cortex is hydrolyzed, soluble hexosamine is lost, refractility decreases, and net adenosine triphophate (ATP) synthesis increases. Small acid-soluble proteins (SASPs), relatively small (from about 12,000 to about 15,000 daltons) proteins, are degraded during germination to provide amino acids for the germinating spore. Generally within only the first few minutes of spore germination, from about ten to about twenty percent of the spore proteins (SASPs) in Bacillus are degraded to amino acids. A tetrameric protease with high specificity for the protein sequences cleaves the SASPs between the glutamine and phenylalanine or isoleucine resides during germination. The later stages of germination are characterized by the activation of specific amidases (cortex-lytic enzymes) that decompose the spore peptidologlycan. Hydrolysis of spore peptidoglycan by spore cortex-lytic enzymes is a significant step in germination. However, there is little known about the mechanism by which the cortex is hydrolyzed during germination and autolytic enzymes are involved (see for example, Nicholson, W. L. and P. Setlow, 1990, Sporulation, germination and outgrowth, Molecular Biological Methods for Bacillus, C. R. Harwood and S. M. Cutting, eds., John Wiley and Sons: New York; pp. 391-429).

After contact of the spores by the germination composition, germination of the spores occurs within a time period that promote functional cleanup of a contaminated area. Preferably, in circumstances that benefit by rapid decontamination, such germination is caused within a sufficiently abbreviated time period for applying the decontaminant and rapidly removing the disinfected spores. Such germination time periods include any appropriate time, such as within 24 hours, less than 4 hours, less than 1 hour, less than fifteen minutes or other time periods that promotes usefulness of areas or articles for their intended purpose. For example, a time period of 24 hours may be sufficient for medical instrumentation that can be placed off-line during decontamination. However, it may be desirable to complete spore germination of spores contaminating areas adjacent to military command centers within less than four hours to allow continued use with minimal interruption during high states of readiness, such as combat operations. Contamination of other areas may necessitate spore germination of less than fifteen minutes, such as operational flight decks on aircraft carriers or other operational environments that have an immediate need for use. It is desirable that the spores are decontaminated within fifteen minutes from application of the decontaminant in wartime situations. As previously stated, Bacillus subtilis spores typically germinate within approximately 20 to 30 minutes from the addition of the germinants to RNA synthesis, but this time period can vary widely even within a single spore population. As a result, in situations that permit additional time periods of decontamination, such as those situations other than immediate response equipment used in military readiness or rescue operations, the application of the germination solution concurrently, before or after the decontaminant (such as during repeated applications of the germination composition/decontaminant solution) for periods of time longer than fifteen minutes may be desirable, and more effective. For example, a time frame of fifteen minutes or less may not be necessary for the decontamination of medical instruments; thus a longer time of exposure may be a more suitable protocol for the decontamination of such equipment. In addition to the factors previously identified, determination of the necessary time period for germination of the spores in a particular area or for a particular object include without limitation, for example, an analysis of the type of area or article, the methods available for the application of the germination composition, the usefulness of the type and amount of disinfectant, particularly with regard to cleanup of non-spore contamination, additional components available for incorporation within the germination composition, the sequence of application of the germination composition and the disinfectant, limitations of concentrations of the components within the germination composition for particular environments, whether the type of germination composition to achieve a given specified time period of germination is desirable for use within a given area or on a given article, etc. Such variables for determination of the time period of germination are determinable by one of ordinary skill in the art of spore decontamination of the contaminated area or article in light of the teachings herein.

The spore germination composition comprises dipicolinic acid (DPA) and calcium ions (Ca²⁺), at appropriate concentrations for effective germination of spores. Dipicolinc acid is also known a pyridine-2,6-dicarboxylic acid, having the molecular formula of C₇H₅NO₄. Calcium ions may be supplied from such compounds as calcium chloride, which may be anhydrous or hydrated, such as calcium chloride dihydrate (CaCl₂.2H₂O), or calcium chloride anhydrous and calcium chloride dehydrated, both having the molecular formula CaCl₂. The relative amounts of dipicolinic acid and calcium chloride are calculated for particular environments, applications and/or disinfectants for increasing the germination of the greatest number of spores within a contaminated area, and decreasing the time period of germination of those spores. Preferably, a one-to-one ratio of calcium ions to dipicolonic acid is used.

Amounts of dipicolinic acid includes any effective amount of dipicolinic acid for spore germination, and may range from about 0.1% by weight DPA to about 99.9% by weight DPA of the germination composition, with preferred amounts of from about 0.8% by weight to about 5% by weight dipicolinic acid of the total spore germination composition, with most preferred amounts of about 1% by weight DPA. Preferred concentrations of the dipicolinic acid range from about 10 mM to about 150 mM dipicolinic acid, more preferably from about 50 mM to about 90 mM dipicolinic acid, and most preferably from about 60 mM to 80 mM dipicolinic acid of the germination composition. Generally, the amount of acidity is minimized, and the amount of DPA used should be that which effectively triggers enzymes 1 G within the spores to effectuate germination.

Amounts of calcium ion includes any effective amount of calcium ion for spore germination. Preferably calcium chloride is used, and may range in concentrations of from about 1 mM or more, with preferred concentrations of the calcium chloride ranging from about 10 mM to about 150 mM calcium chloride, more preferably from about 50 mM to about 90 mM calcium chloride, and most preferably from about 60 mM to 80 mM calcium chloride, of the germination composition. Preferably, a one-to-one ratio of calcium ions to dipicolonic acid is used.

Additional components may be incorporated within the germination composition as desired, particularly for enhancing effective germination, solubility, stability and/or other desirable properties of the germination composition. A medium to facilitate application of the dipicolinic acid and calcium chloride is preferably used within germination composition, with the medium preferably comprising an aqueous composition. Preferably the aqueous composition comprises water in an amount of from about 20% w/w or more, with more preferred amounts of from about 50% w/w to about 98% w/w water to the total germination composition. Other additional components may be included, such as one or more surfactants, preferably anionic or nonionic surfactants. These surfactants preferably include long-chain surfactants having at least one carbon chain of from about six carbon atoms or more. The amount of surfactant within a particular germination composition is determinable by those skilled in the art in light of the teachings herein, with preferred amounts of the surfactant generally ranging from about 5% w/w to about 15% w/w of the total spore germination composition.

Selection of the type of surfactant preferably includes a functional determination for the desired properties of the germination composition. Surfactants may be added to the germination solution for improved characteristics, such as solubility of the dipicolinic acid within a given solution. Nonionic surfactants generally improve germination effectiveness of the germination composition, with representative nonionic surfactants including such non-limiting examples as sugar surfactants and amine oxides, with particularly preferred nonionic surfactants including amines oxides, and sugar surfactants, such as alkyl polyglycosides or alkyl polysaccharide ethers (with at least eight carbon atoms). Anionic surfactants appear to improve compatibility of the germination composition when it is incorporated into a decontaminant. Representative anionic surfactants generally including such non-limiting examples as diphenyl sulfonate derivatives.

Water may be included within the germination composition. A neutral to acidic pH is preferred, with a pH range of from about 2 to about 8 more preferred, and a pH range of from about 6 to about 8 most preferred. Additional components may be incorporated within the germination composition that do not interfere with the functionality of the germination composition. The present invention is applicable for decontamination of biological spores, particularly biological spores that comprise bacterial endospores. Typical spores susceptible to the germination composition of the present invention include bacteria belonging to the genus Bacillus and Clostridium. Representative endospores include almost all Bacillus and Clostridium species, including, but not limited to Bacillus subtilis, Bacillus anthracis and Bacillus globigii.

Application of the germination composition may include any desirable means for a given area or article, as determinable by those skilled in the art. Application includes washing application systems, sprayers, brushes, mops and other like applicators, useful for a given area or article. Preferably, the application of the germination composition occurs in a manner that allows for the most effective concentration of germination composition to contact contaminant spores for a sufficient period of time for effective germination. The area may include desk tops, floors and hallways, flight decks, buildings, vehicles surfaces and other like structures. Articles include objects and devices such as mechanical devices, plates, silverware, bowls, tools, computers, vehicles, electronics, and the like. Surfaces may include vertical or horizontal hard surfaces, equipment, clothing, personal articles, etc. Spore germination, however, is generally avoided for spores that are in contact with, or that will potentially contact, persons within or transiting the contaminated area, or that will enter the contaminated area.

The decontaminating solution may include any type of known decontaminant that remains effective against biological spore contamination, and that does not interfere with the germination composition of the present invention. Generally, the decontaminant includes a liquid decontaminant, or decontaminant solution, that permits effective decontaminating or disinfecting of a particular surface or area. Additionally, more than one decontaminant, or repeated applications of the one or more decontaminants, may be used ensure cleanup of the contaminated area or surface. Preferably the decontaminant solution is applied to the contaminated area in the same manner as the germination composition, which generally minimizes the complexity of the decontamination; however, where particular circumstances permit, different types of application of the germination composition and decontaminant may be desired, such as application of the germination composition under a decontaminant of a purge of toxic gas.

The decontaminant used in conjunction with the germination composition for disinfecting biological spores includes a type and amount of decontaminant that is effective for decontaminating the germinated spores within a given environment. Selection of the appropriate decontaminant is determinable by such factors as use concurrently or sequentially with the germination composition, type of spore contamination cleanup needed, type of surface or area to be decontaminated, environmental conditions of the cleanup, and other such criteria that are determinable by those skilled in the area of decontamination. When the disinfectant is used after the application of the germination composition, preferably the decontaminant includes amine oxide surfactant(s) and peroxygen compound. When used concurrently with the germination composition, the decontaminant comprises components that do not substantially interfere with the mechanism of action of the germination composition to cause the spores to germinate. When the decontaminant is used concurrently with the germination composition, preferably the decontaminant also includes amine oxide surfactant(s) and peroxygen compound.

Decontaminants may include optional components such as catalysts, surfactants, sodium carbonate, sodium hydroxide, water, enzymes, and the like. Enzymes are particularly useful for decontaminating vegetative bacteria and may decontaminate some spores when applied with the germination solution. Preferred amounts of enzymes include approximately 1 mg/ml with activity of from about 10 to about 20 units per mg.

Effectiveness of the disinfectant of the present invention occurs with increases of biological spore “kills” with the use of the germination composition over non-use. Preferably, an effective kill is variable on the original number of spores within a contamination, such as a 90% effectiveness (kill) against a concentration of 10⁸ spores/ml, and more preferably an effectiveness of 90% against a concentration of 10⁸ spores/ml, with a most preferred decontamination of from about three or more logarithmic reductions of live spores. Most preferably, the decontamination reduces the spore concentration to a level that renders the once hazardous contaminated area or surface no longer hazardous.

Spores are killed when they are rendered harmless, i.e., no longer hazardous, to a particular living organism, particularly a human. Depending on the circumstances, spore decontamination may be desirable against spores that affect other mammals, animals or plants. These areas are contacted with the germination composition in a manner to best facilitate germination of the contamination for the environmental conditions that exist. For example, a space heater may be used to heat indoor areas prior to the application of the germination composition to increase ambient and surface area temperatures and facilitate germination of contaminate spores in those areas at optimal temperatures. As germination generally increases with moderate heat, as do most chemical reactions, the retention or application of moderate heat into the contaminate surface in combination with the germination composition, if possible, is generally desirable. Additionally, heat activating bacterial spores, prior to application of the germination composition, promotes more uniform germination of the spore population. Preferred amounts of heat include temperatures of from about 4° C. to about 70° C.

Application of the decontaminating solution occurs in a manner to minimize any interference of the germination composition by the decontaminating solution. Typically, the decontaminating solution is applied after the germination composition has been applied, and at a time that has allowed the germination composition to cause germination of the contaminate spores. The application of the germination composition prior to the application of the decontaminating solution generally ensures that the decontaminating solution does not mitigate the germination properties of the germination composition. Mitigation may occur through several processes which, in addition to chemical neutralization of the germination composition, includes physically blocking the germination composition from sufficiently contacting the spores, diluting the germination composition to an ineffective concentration, removal of the germination composition prior to germination, etc. Mitigation may also occur when the germination composition is applied in conjunction with a disinfectant that rapidly removes the spore coat or cortex in such a manner as to prevent the germination composition from initiating the germination response. Additionally, application of the germination composition should be accomplished in a manner that minimizes any possible interference with the decontaminating solution. Concurrent application of the germination composition and the decontaminant may be done, and is preferably done, when the selected germination composition and disinfectant do not interfere with each other. Accordingly, the method for decontaminating contamination containing biological spores may include contacting the spore contamination with a spore germination composition in an application that occurs prior to the step of applying a decontaminating solution to kill the germinated spores, application of the spore germination composition concurrently, or simultaneously, with the step of applying a decontaminating solution to kill the germinated spores, or may when desired, after the application of the decontaminating solution (such as during repeated applications of the germination composition/decontaminant solution). Preferably, the application of the germination composition onto an area of spore contamination occurs concurrently with or prior to the application of the decontaminating solution, and for convenience of use, most preferably, the application of the spore germination composition occurs concurrently with the decontaminating solution, although application of the spore germination composition before the application of the decontaminating solution may be desirable particularly for combinations of spore germination compositions and decontaminating solutions that interact and/or interfere with each other.

### EXAMPLE 1

An initial concentration of 1.0 mg dry Bacillus globigii spores (equivalent to 1×10⁸ viable CFU/ml according to Industry and Dugway Life Sciences standard which was independently verified) was weighed out in a two ml microcentrifuge tube. To verify that 1.0 mg of Bacillus globigii dry spores was equivalent to 108 CFU/ml, the following protocol was followed. In triplicate, 1.0 mg of Bacillus globigii dry spores was weighed out into two milliliter microcentrifuge tubes. One milliliter of phosphate buffered saline (PBS) was added to each sample. Samples were thoroughly vortexed. From each microcentrifuge tube, 100 μl were withdrawn and pipetted into a 1.7 ml microcentrifuge tube containing 900 μl of PBS. The tube was well vortexed and a new pipette tip was used to carry out the next dilution. The process was repeated out to the appropriate dilution (1/10⁸ for 10⁸ CFU/ml). All dilutions, as well as the initial test solution, were plated in duplicate on Luria-Bertani agar. The plates were incubated overnight at 37° C. Colonies that had grown overnight were counted and recorded. Results indicated that 1.0 mg of Bacillus globigii spores was equivalent to approximately 10⁸ CFU/ml.

For each replication, one ml of each decontamination solution or PBS (control) was added to a two ml tube containing a sterilized magnetic stir bar. The candidate decontamination solutions and control were tested in triplicate for effectiveness against B. globigii spores.

The following Table 1 shows the make-up of the decontamination solutions of Example 1:

TABLE 1

| Components | 1A | | 1B | | 1C | |
|---|---|---|---|---|---|---|
| non-ionic, alkyl polyglycoside surfac- | 235.7 | mg | 235.7 | mg | 235.7 | mg |
| tant: Glucopon 225 DK | | | | | | |
| peroxygen compounds: | 357.0 | mg | 357.0 | mg | 357.0 | mg |
| sodium nonanoyloxybenzenesulfonate | | | | | | |
| tert-butyl hydrogen peroxide | 558 | μl | 558 | μl | 558 | μl |
| water | 2786 | μl | 2786 | μl | 2786 | μl |

| | | | | | |
|---|---|---|---|---|---|
| dipicolinic acid | NA | 30.1 | mg | 30.1 | mg |

| | | | | |
|---|---|---|---|---|
| calcium chloride | NA | NA | 26.5 | mg |

As mentioned previously, one milliliter of the above solutions or a control (PB S) was added to each of three replicate tubes and allowed to stir for a period of fifteen minutes.

The decontamination solutions and control were placed on a magnetic stir plate and allowed to stir for 15 minutes at room temperature. After mixing for the selected time, one ml of 33% by weight sodium metabisulfite (Na₂S₂O₅) solution was added to reduce/neutralize the candidate decontamination solutions. The magnetic stir bars were then removed and the solutions were centrifuged at 14,000 rpm for one minute. The supernatant was removed from each tube and the B. globigii pellet was resuspended in one ml of PBS by vortexing. The tubes were centrifuged again at 14,000 rpm for one minute. The resulting B. globigii pellet was “washed” and resuspended in PBS a total of three times. After the third wash and resuspension, serial dilutions were made from this resuspended pellet. From each test tube, 100 μl were withdrawn and pipetted into a 1.7 ml microcentrifuge tube containing 900 μl of PBS. The tube was well vortexed and a new pipette tip was used to carry out the next dilution. The process was repeated out to the appropriate dilution (1/10⁸ for 10⁸ CFU/ml). All dilutions, as well as the initial resuspended pellet, were plated in duplicate on Luria-Bertani agar. The plates were incubated overnight at 37° C. Colonies that had grown overnight were counted and recorded as an indication of efficacy of the candidate decontamination solutions, with the results shown in Table A. Decontamination solutions appeared to work best when both CaCl₂ and dipicolinic acid were incorporated into the solution versus the application of only one component of the germination composition.

### EXAMPLE 2

An initial concentration of 1.0 mg dry Bacillus globigii spores (equivalent to 1×10⁸ viable CFU/ml according to Industry and Dugway Life Sciences standard, and independently verified) was weighed out in a two ml microcentrifuge tube. For each replication, 0.5 ml of each test solution or PBS (control) was added to a two ml tube containing a sterilized magnetic stir bar and 1.0 mg dry Bacillus globigii spores. The candidate decontamination solutions and control (PBS) were tested in triplicate for effectiveness against B. globigii spores.

The following Table 2 shows the make-up of the germination solutions of Example

TABLE 2

| Components | A | | B | |
|---|---|---|---|---|
| anionic, diphenyl sulphonate derivative sur- | 469.7 | mg | 469.7 | mg |
| factant: Dowfax 8390 | | | | |
| water | 1071.4 | μl | 1071.4 | μl |
| dipicolinic acid | 15.0 | mg | 30.1 | mg |
| calcium chloride | 13.2 | mg | 26.5 | mg |

The samples were placed on a magnetic stir plate and allowed to stir for 15 minutes at room temperature. Concurrently, decontaminating solution #2 containing 357.0 mg sodium nonanoyloxybenzenesulfonate, 558 μl tert-butyl hydrogen peroxide and 942 μl water was allowed to stir for 15 minutes. Then, 0.5 ml of decontaminating solution #2 was added to the samples containing 0.5 ml of test solutions A and B, resulting in test solutions #2A and #2B. To the control, an additional 0.5 ml of PBS was added. Samples were briefly vortexed, then placed on a magnetic stir plate and allowed to stir for 15 minutes at room temperature. The samples were then centrifuged at 14,000 rpm for one minute. The supernatant was removed and all solutions were neutralized with one ml of 33% by weight sodium metabisulfite (Na₂S₂O₅) solution. The magnetic stir bars were then removed and the solutions were centrifuged at 14,000 rpm for one minute. The supernatant was removed from each tube and the B. globigii pellet was resuspended in one ml of PBS by vortexing. The tubes were centrifuged again at 14,000 rpm for one minute. The resulting B. globigii pellet was washed and resuspended in PBS a total of three times. After the third wash and resuspension, serial dilutions were made from this resuspended pellet. From each test tube, 100 μl were withdrawn and pipetted into a 1.7 ml microcentrifuge tube containing 900 μl of PBS. The tube was well vortexed and a new pipette tip was used to carry out the next dilution. The process was repeated out to the appropriate dilution (1/10⁸ for 10⁸ CFU/ml). All dilutions, as well as the initial resuspended pellet, were plated in duplicate on Luria-Bertani agar. The plates were incubated overnight at 37° C. Colonies that had grown overnight were counted and recorded as an indication of efficacy of the candidate decontamination solutions.

Results are shown in Table B. Results indicate that the efficacy of the decontamination solutions may be increased with various concentrations of germinating compositions. The effect was particularly evident here, when studying a decontamination solution containing an anionic surfactant (Dowfax 8390) and peroxygen compounds (sodium nonanoyloxybenzenesulfonate and tert-butyl hydrogen peroxide). Additionally, results indicate that it is possible to just add the decontaminant to the germination composition, without removing the germination composition or washing, and still have a favorable effect on the performance of the decontaminant.

### EXAMPLE 3

The same procedures as in Example 2 were followed, changing only the composition of the solutions. In Example 3, the initial test solutions were comprised of the following shown in Table 3, below:

TABLE 3

| Components | A | | B | |
|---|---|---|---|---|
| anionic, diphenyl sulphonate derivative sur- | 469.7 | mg | 469.7 | mg |
| factant: Dowfax 8390 | | | | |
| water | 1071.4 | μl | 1071.4 | μl |

| | | | |
|---|---|---|---|
| dipicolinic acid | NA | 30.1 | mg |
| calcium chloride | NA | 26.5 | mg |

0.5 ml of each test solution listed above (and a PBS control) was added in triplicate to each sample and allowed to stir for 15 minutes. Concurrently, decontaminating solution #3 containing 357.0 mg sodium nonanoyloxybenzenesulfonate, 558 μl tert-butyl hydrogen peroxide, 6.3 mg accelerator catalyst (a product of Hickson and Welch, Ltd, West Yorkshire, WF 102JF, UK, product code SDS0708) and 942 μl water was allowed to stir for 15 minutes. After 15 minutes, 0.5 ml of decontaminating solution #3 was added to the samples containing test solutions A and B, resulting in test solutions #3A and #3B. Samples were then allowed to stir for 15 minutes. Results are shown in Table B. The addition of a catalyst (compared to solution #2 in Example 2) improved the efficacy of the decontaminant, with the germination composition further improving the efficacy of the decontaminant, and as such remained a useful addition to the decontaminant.

### EXAMPLE 4

The same procedures as in Example 1 were followed, changing only the composition of the decontamination solutions. In Example 4, decontamination solution #4A comprised 1.0 mg/ml proteinase K (32 mg/unit) in water. Decontamination solution #4B comprised 104.6 mg/ml of an alkyl polysaccharide ether nonionic surfactant, Glucopon 225 DK, 1.0 mg/ml enzyme (proteinase K), 10.0 mg/ml dipicolinic acid, 8.8 mg/ml calcium chloride and 928.6 μl/ml of water. One milliliter of each decontamination solution or control (PBS) was added to the tubes containing Bacillus globigii and allowed to stir for a period of 15 minutes (see protocol outlined in Example 1). Results are shown in Table A. The results indicate that the addition of the germination composition to a solution containing some enzymes may enable the previously ineffective solution to have some limited efficacy against endospore forming bacteria.

### EXAMPLE 5

The same procedures as in Example 1 were followed, changing only the composition of the decontamination solutions and increasing the time of exposure to 30 minutes.

The following Table 5 shows the make-up of the decontamination solutions of Example 5:

TABLE 5

| Components | 5A | | 5B | | 5C | | 5D | |
|---|---|---|---|---|---|---|---|---|
| non-ionic, | 1440.0 | mg | 1440.0 | mg | 1440.0 | mg | 1440.0 | mg |
| amine oxide | | | | | | | | |
| surfactant: | | | | | | | | |
| Barlox 10S | | | | | | | | |
| non-ionic, | 464.0 | mg | 464.0 | mg | 464.0 | mg | 464.0 | mg |
| amine oxide | | | | | | | | |
| surfactant: | | | | | | | | |
| Damox 1010 | | | | | | | | |
| peroxygen: | 14.0 | mg | 14.0 | mg | 147.0 | mg | 147.0 | mg |
| tetraacetyl- | | | | | | | | |
| ethylene- | | | | | | | | |
| diamine | | | | | | | | |
| peroxygen: | 14.0 | mg | 14.0 | mg | 247.0 | mg | 247.0 | mg |
| urea hydrogen | | | | | | | | |
| peroxide | | | | | | | | |
| sodium car- | 42.0 | mg | 42.0 | mg | 42.0 | mg | 42.0 | mg |
| bonate | | | | | | | | |
| accelerator | 2288 | μl | 2288 | μl | 2288 | μl | 2288 | μl |
| catalyst | | | | | | | | |
| (438.5 mg/ | | | | | | | | |
| 200 ml H₂O) | | | | | | | | |

| | | | | | | |
|---|---|---|---|---|---|---|
| dipicolinic acid | NA | 40.1 | mg | NA | 40.1 | mg |
| calcium chlo- | NA | 35.3 | mg | NA | 35.3 | mg |
| ride | | | | | | |

One milliliter of each decontamination solution or control (PBS) was added to the tubes containing Bacillus globigii and allowed to stir for a period of 15 minutes (see protocol outlined in Example 1).

Results are shown in Table A. The addition of the germination composition and decontamination solution for longer periods of time than 15 minutes may improve the efficacy of the decontamination. Additionally, the use of the germination composition allows for lower concentrations of the decontaminant (an amine oxide surfactant solution containing peroxygen compounds tetraacetylethylenediamine and urea hydrogen peroxide), while resulting in a greater spore kill.

### EXAMPLE 6

The same procedures as in Example 1 were followed, changing only the composition of the decontamination solutions.

The following Table 6 shows the make-up of the decontamination solutions of Example 6:

TABLE 6

| Components | 6A | 6B | 6C | 6D |
|---|---|---|---|---|
| 30% hydrogen peroxide | 500 μl/ml | 500 μl/ml | 100 μl/ml | 100 μl/ml |
| water | 100 μl/ml | NA | 900 μl/ml | NA |
| stock solution (60 mM | NA | 500 μl/ml | NA | 900 μl/ml |
| DPA & CaCl₂) | | | | |

Results are shown in Table A. The results of Example 6 indicate that improved efficacy may not occur where the germination composition and the decontaminant interfere with each other. This may occur where the decontaminant interferes with the germination itself or the mechanism of action of the germination solutions, or where the germination solution interferes with the decontaminant, such as reducing the availability of hydrogen peroxide. It is further believed that since the hydrogen peroxide degrades the spore cortex, the addition of the germination composition may not greatly improve a solution based predominately on hydrogen peroxide.

### EXAMPLE 7

Procedures were followed as in Example 1, changing only the composition of the decontamination solution.

The following Table 7 shows the make-up of the decontamination solutions of Example 7:

TABLE 7

| Components | 7A | | 7B | |
|---|---|---|---|---|
| non-ionic, amine oxide surfactant: | 1440.0 | mg | 1440.0 | mg |
| Barlox 10S | | | | |
| non-ionic, amine oxide surfactant: | 464.0 | mg | 464.0 | mg |
| Damox 1010 | | | | |
| ε-n,n-phthaloylaminoperoxy caproic acid | 332.1 | mg | 332.1 | mg |
| sodium carbonate | 42.0 | mg | 42.0 | mg |
| accelerator catalyst (438.5 mg/200 ml H₂O) | 2288 | μl | 2288 | μl |

| | | | |
|---|---|---|---|
| dipicolinic acid | NA | 40.1 | mg |
| calcium chloride | NA | 35.3 | mg |

One milliliter of each decontamination solution or control (PBS) was added to the tubes containing Bacillus globigii and allowed to stir for a period of 15 minutes (see protocol outlined in Example 1). Results are in Table A. Results indicate that the addition of the germination composition to a decontaminant containing a free peracid, particularly as in e-n,n-phthaloylaminoperoxy caproic acid, will improve the efficacy of the decontaminant.

TABLE A
Logarithmic reduction in 10⁸ Bacillus globigii colony forming units per
milliliter (CFU/ml) after 15 minutes exposure (or 30 minutes where
designated) to candidate decontamination solutions for
the examples listed

| Solution | Logarithmic Reduction | |
|---|---|---|
| Number | in CFU/ml | Example |
| 1A | 1 | Example 1 |
| 1B | 1 | |
| 1C | 2 | |
| 4A | 0 | Example 4 |
| 4B | <1 | |
| 5A | 1 | Example 5 |
| 5B | 3 | (30 minutes) |
| 5C | 8 | |
| 5D | 8 | |
| 6A | 6 | Example 6 |
| 6B | 2 | |
| 6C | 3 | |
| 6D | 2 | |
| 7A | 2 | Example 7 |
| 7B | 3 | |

TABLE B
Logarithmic reduction in 10⁸ Bacillus globigii colony forming units per
milliliter (CFU/ml) after 15 minutes exposure to the germination
composition then 15 minutes exposure to the germination
composition plus the candidate decontamination
solutions for the examples listed

| Solution | Logarithmic Reduction | |
|---|---|---|
| Number | in CFU/ml | Example |
| 2A | 1 | Example 2 |
| 2B | 2 | |
| 3A | 2 | Example 3 |
| 3B | 3 | |

### EXAMPLE 8

The same procedures as in Example 1 were followed, changing only the composition of the decontamination solutions.

The following Table 8 shows the make-up of the decontamination solutions of Example 8:

TABLE 8

| Components | 8A | | 8B | |
|---|---|---|---|---|
| non-ionic, amine oxide surfactant: | 1440.0 | mg | 1440.0 | mg |
| Barlox 10S | | | | |
| non-ionic, amine oxide surfactant: | 464.0 | mg | 464.0 | mg |
| Damox 1010 | | | | |
| peroxygen: tetraacetylethylenediamine | 80.5 | mg | 80.5 | mg |
| peroxygen: urea hydrogen peroxide | 81.5 | mg | 81.5 | mg |
| sodium carbonate | 42.0 | mg | 42.0 | mg |
| accelerator catalyst (438.5 mg/200 ml H₂O) | 2288 | μl | 2288 | μl |

| | | | |
|---|---|---|---|
| dipicolinic acid | NA | 40.1 | mg |
| calcium chloride | NA | 35.3 | mg |

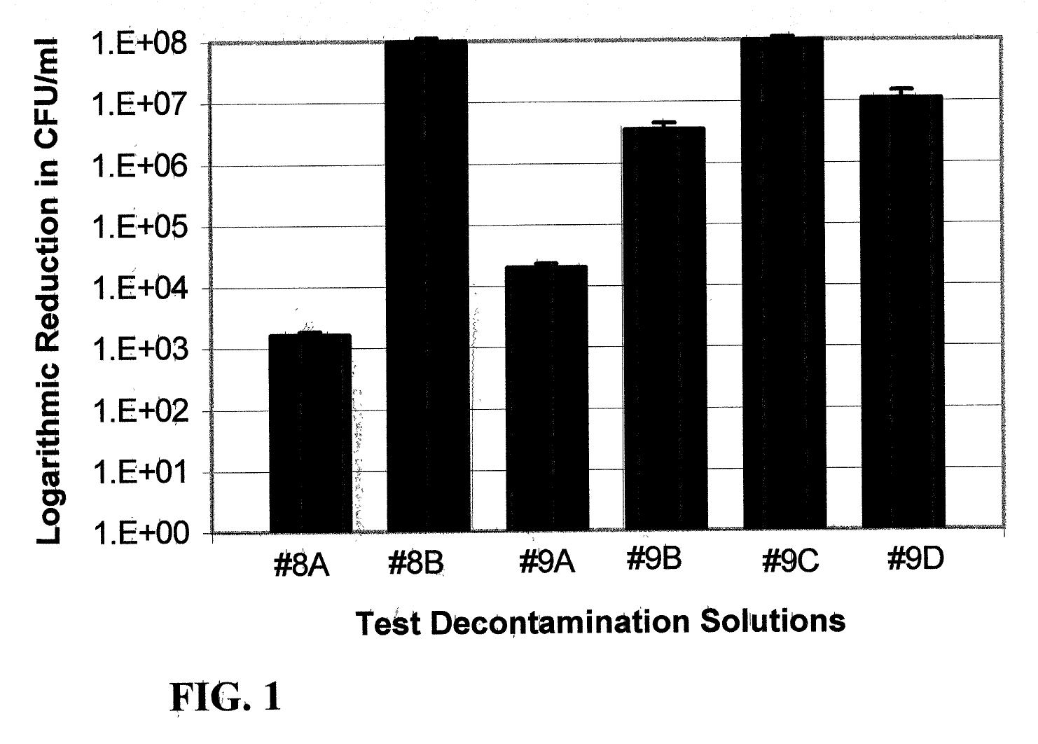

One milliliter of each decontamination solution or control (PBS) was added to the tubes containing Bacillus globigii and allowed to stir for a period of 15 minutes (see protocol outlined in Example 1). Note that the components of the decontaminant in this Example 8 were the same as in Example 5, changing only exposure time and reducing the concentration of peroxygen compounds. Results are shown in FIG. 1. The addition of the germination composition to a decontaminant containing amine oxide surfactant and peroxygen compounds (such as tetraacetylethylenediamine and urea hydrogen peroxide) may allow for lower concentrations of the decontaminant to be utilized and still have an effective spore kill.

### EXAMPLE 9

The same procedures as in Example 1 were followed, changing only the composition of the decontamination solutions.

The following Table 9 shows the make-up of the decontamination solutions of Example 9:

TABLE 9

| Components | 9A | | 9B | | 9C | | 9D | |
|---|---|---|---|---|---|---|---|---|
| non-ionic, | 1440.0 | mg | 1440.0 | mg | 1440.0 | mg | 1440.0 | mg |
| amine oxide | | | | | | | | |
| surfactant: | | | | | | | | |
| Barlox 10S | | | | | | | | |
| non-ionic, | 464.0 | mg | 464.0 | mg | 464.0 | mg | 464.0 | mg |
| amine oxide | | | | | | | | |
| surfactant: | | | | | | | | |
| Damox 1010 | | | | | | | | |
| 15% peracetic | 318.6 | μl | 318.6 | μl | 424.8 | μl | 424.8 | μl |
| acid | | | | | | | | |
| sodium car- | 42.0 | mg | 42.0 | mg | 42.0 | mg | 42.0 | mg |
| bonate | | | | | | | | |
| accelerator | 1969.4 | μl | 1969.4 | μl | 1863.2 | μl | 1863.2 | μl |
| catalyst | | | | | | | | |
| (438.5 mg/ | | | | | | | | |
| 200 ml H₂O) | | | | | | | | |

| | | | | | | |
|---|---|---|---|---|---|---|
| dipicolinic acid | NA | 40.1 | mg | NA | 40.1 | mg |
| calcium | NA | 35.3 | mg | NA | 35.3 | mg |
| chloride | | | | | | |

One milliliter of each decontamination solution or control (PBS) was added to the tubes containing Bacillus globigii and allowed to stir for a period of 15 minutes (see protocol outlined in Example 1). Results are shown in FIG. 1. Results indicated that this particular peroxygen (15% peracetic acid) had stability problems that should be overcome before benefit can be seen from the incorporation of the germination composition.

### EXAMPLE 10

An initial concentration of 1.0 mg dry Bacillus globigii spores (equivalent to 1×10⁸ viable CFU/ml according to Industry and Dugway Life Sciences standard) was weighed out in a two ml microcentrifuge tube. For each replication, one ml of each decontamination solution or PBS (control) was added to a two ml tube containing a sterilized magnetic stir bar and 1.0 mg dry Bacillus globigii spores. The candidate germination solutions and control in combination with decontamination solutions were tested in triplicate for effectiveness against B. globigii spores. Test germination solution A contained 1% by weight dipicolinic acid (DPA) and 0.8% by weight calcium chloride, with the balance ofwater and having pH 7. One 5 ml of test solution A was added to each of three samples containing 1.0 mg dry Bacillus globigii spores. The samples were placed on a magnetic stir plate and allowed to stir for 120 minutes at room temperature. The samples were then centrifuged at 14,000 rpm for one minute. The supernatant was discarded and one ml of 6% sodium hypochlorite (decontaminant) was added to each sample containing solution A (called Sample #10A). Samples were then allowed to stir for 15 minutes. 6% hypochlorite also was added to three samples containing 11.0 mg dry Bacillus globigii spores (called Sample#10B). Sample #10B was allowed to stir for a period of 15 minutes. (Note: Spores tested with #10A received benefit of the germination composition prior to exposure to the 6% hypochlorite solution, whereas spores in solution #10B received no prior treatment.)

After exposure to the 6% sodium hypochlorite solution for 10 minutes, all solutions were neutralized with 33% by weight of 1 ml of sodium metabisulfite (Na₂S₂O₅) solution. The magnetic stir bars were then removed and the solutions were centrifuged at 14,000 rpm for one minute. The supernatant was removed from each tube and the B. globigii pellet was resuspended in one ml of PBS by vortexing. The tubes were centrifuged again at 14,000 rpm for one minute. The resulting B. globigii pellet was washed and resuspended in PBS a total of three times. After the third wash and resuspension, serial dilutions were made from this resuspended pellet. From each test tube, 100 μl were withdrawn and pipetted into a 1.7 ml microcentrifuge tube containing 900 μl of PBS. The tube was well vortexed and a new pipette tip was used to carry out the next dilution. The process was repeated out to the appropriate dilution (1/10⁸ for 10⁸ CFU/ml). All dilutions, as well as the initial resuspended pellet, were plated in duplicate on Luria-Bertani agar. The plates were incubated overnight at 37° C. Colonies that had grown overnight were counted and recorded as an indication of efficacy of the candidate decontamination solutions.

The decontamination methodology used with #10A resulted in a 5 logarithmic reduction in CFU/ml, while the test solution #10B resulted in a 4 logarithmic reduction in CFU/ml, indicating the improved efficacy with the prior treatment of the germination composition.

The foregoing summary, description, and examples of the present invention are not intended to be limiting, but are only exemplary of the inventive features which are defined in the claims.

What is claimed is:

1. A method for decontaminating contamination containing biological spores, comprising the steps of:
contacting the contamination with a spore germination composition comprising dipicolinic acid and calcium ions effective to cause germination of the spores; and, applying a decontaminating solution to kill the germinated spores.

2. The method of claim 1, wherein the step of contacting the contamination with the spore germination composition effective to cause germination of the spores simultaneously with the step of applying a decontaminating solution to kill the germinated spores.

3. The method of claim 1, wherein the step of contacting the contamination with the spore germination composition effective to cause germination of the spores occurs prior to the step of applying a decontaminating solution to kill the germinated spores.

4. The method of claim 1, wherein the spore germination composition comprises from about 50 mM to about 90 mM dipicolinic acid.

5. The method of claim 1, wherein the calcium ions comprise calcium chloride.

6. The method of claim 5, wherein the spore germination composition comprises from about 50 mM to about 90 mM calcium chloride.

7. The method of claim 6, wherein the spore germination composition comprises from about 60 mM to about 80 mM calcium chloride.

8. The method of claim 1, wherein the spore germination composition comprises from about 0.8% w/w to about 5% w/w dipicolinic acid of the total spore germination composition.

9. The method of claim 1, wherein the spore germination composition further comprises water.

10. The method of claim 9, wherein the spore germination composition comprises from about 50% w/w to about 98% w/w water.

11. The method of claim 1, wherein the spore germination composition further comprises a surfactant.

12. The method of claim 11, wherein the surfactant is selected from the group consisting of anionic surfactant and nonionic surfactant.

13. The method of claim 11, wherein the surfactant comprises at least one carbon chain of from about six carbon members or more.

14. The method of claim 11, wherein the surfactant comprises from about 5% w/w to about 15% w/w of the total spore germination composition.

15. The method of claim 1, wherein the decontaminating solution comprises enzymes.

16. The method of claim 1, wherein the decontaminating solution comprises a peroxygen compound.

17. A germination composition for decontaminating biological spores, comprising dipicolinic acid and calcium ions.

18. The germination composition of claim 17, wherein the calcium ions are supplied by calcium chloride.

19. A decontaminated surface made by the process comprising the steps of:
contacting a surface with a spore germination composition comprising dipicolinic acid and calcium ions effective to cause germination of the spores; and, applying a decontaminating solution to kill the germinated spores.

20. A method for decontaminating a chemical-biological agent comprising the steps of:
solubilizing the chemical-biological agent with a microemulsion having a plurality of surfactants; and,
decontaminating the solubilized chemical-biological agent with a peracid.
